(19) United States
(12) Patent Application Publication
(10) Pub. No.: US 2024/0175866 A1
(43) Pub. Date: May 30, 2024

(54) Method for Detecting Coronavirus Infection

(71) Applicants: NATIONAL UNIVERSITY CORPORATION KUMAMOTO UNIVERSITY, Kumamoto-shi (JP); SHIMADZU CORPORATION, Kyoto-shi (JP); AiSTI SCIENCE Co., Ltd., Wakayama-shi (JP)

(72) Inventors: Kazuhito TOMIZAWA, Kumamoto-shi (JP); Yu NAGAYOSHI, Kumamoto-shi (JP)

(73) Assignees: NATIONAL UNIVERSITY CORPORATION KUMAMOTO UNIVERSITY, Kumamoto-shi (JP); SHIMADZU CORPORATION, Kyoto-shi (JP); AiSTI SCIENCE Co., Ltd., Wakayama-shi (JP)

(21) Appl. No.: 18/281,094

(22) PCT Filed: Mar. 9, 2022
(86) PCT No.: PCT/JP2022/010333

§ 371 Date: Sep. 8, 2023

(30) Foreign Application Priority Data
Mar. 10, 2021 (JP) 2021-038698
Jun. 1, 2021 (JP) 2021-092199

(51) Int. Cl. G01N 33/53

(52) U.S. Cl. CPC G01N 33/5308; G01N 2800/50; G01N 2800/52

(57) ABSTRACT

An object of the present invention is to provide a method for determining that a subject is at risk of suffering or is suffering from COVID-19. The present invention provides a method for determining whether a subject that is a mammal is at risk of suffering or suffering from COVID-19, the method including a step of detecting the amount of at least one modified nucleoside selected from the group consisting of 6-threonylcarbamoyl adenosine (t⁶A) and 2-thiomethyl,6-threonylcarbamoyl adenosine (ms²t⁶A) in a sample derived from the subject.

### TECHNICAL FIELD

The present invention relates to a method for detecting a subject at risk of suffering or a subject suffering from COVID-19. More specifically, the present invention relates to a method for detecting a subject at risk of suffering or a subject suffering from COVID-19 by using modified nucleosides as targets.

### BACKGROUND ART

The pneumonia of unknown cause reported in Wuhan, Hubei, China in December 2019 was an infection by a novel pathogenic virus called SARS-CoV2 and named COVID-19. The infection has since spread worldwide, resulting in many deaths as well as significant impacts on social and economic activities. Currently, a PCR test and an antigen test are used for diagnosis of the same disease. However, the antigen test has low accuracy, and the PCR test is thus used for definite diagnosis in most cases. The PCR test is highly accurate, but nasopharyngeal swabs, sputum, or saliva is used as a specimen, and there is thus a risk of infection for medical workers and laboratory technicians, and a person who performs the work is forced to bear a mental and physical burden. In addition to the fact that it takes time to find the inspection result, it has been pointed out that preprocessing is often performed manually under the present circumstances, which is complicated. In addition, it is not possible to determine the severity or therapeutic effect by the current inspection method, and it is difficult to evaluate the condition of the patient in a clinical setting. Therefore, there is a demand for a COVID-19 inspection method that is more convenient and enables evaluation of disease.

One of the inventors of the present invention has previously demonstrated and reported the physiological significance in mammals for chemical modifications in ribonucleic acid (RNA). In the process, the inventor found that a nucleic acid once modified is decomposed into nucleosides and excreted extracellularly, and established a technique for comprehensively analyzing modified nucleosides in blood and urine (Non Patent Literature 1). It is known that for these modified nucleosides, some are common regardless of species and others are specific to species.

tRNA is an adaptor molecule that converts genetic information of DNA written by four bases into an amino acid sequence of a protein. tRNAs are subjected to various post-transcriptional modifications, which are not only necessary for tRNA folding and stability, but also play an important role in accurately and efficiently decrypting the genetic code.

N⁶-threonylcarbamoyl adenosine (t⁶A) is a derivative of adenosine and has a chemical structure in which threonine is bonded at the N6 position via a carbonyl group. t⁶A is a modified base present at position 37 of the tRNA that decodes the ANN codon, and is a modified nucleoside that is conserved in almost all organisms and is essential for the growth of many organisms. t⁶A is known to play important roles in various stages of protein synthesis, such as aminoacylation of tRNA, translocation reactions, accurate recognition of codons, and maintenance of reading frames.

2-Thiomethyl,6-threonylcarbamoyl adenosine (ms²t⁶A) has a chemical structure in which the 2-position of adenine of t⁶A is thiomethylated. It has been reported by one of the inventors of the present invention that ms²t⁶A is a modified base present at position 37 of tRNA whose anticodon is UUU, and is biosynthesized from t⁶A by Cdkal1, which is a methylthiotransferase (Non Patent Literature 2).

Modified nucleosides have also been reported to be associated with disease. ms²t⁶A has been reported by one of the inventors of the present invention to be associated with type 2 diabetes (Non Patent Literature 2). However, there is no report on the association of these modified nucleosides with infectious viruses including COVID-19.

### CITATION LIST

### Non Patent Literature

Non Patent Literature 1: Ogawa et al., Molecular Cell 81, 659-674, 2021
Non Patent Literature 2: Wei et al., J Clin Invest. 2011; 121 (9): 3598-3608

### SUMMARY OF INVENTION

### Technical Problem

In one mode, an object of the present invention is to provide a method for detecting a subject at risk of suffering or a subject suffering from COVID-19. In another mode, an object of the present invention is to provide a method for predicting the severity of a patient suffering from COVID-19.

### Solution to Problem

The inventors of the present invention have comprehensively analyzed the modified nucleosides in the blood and urine of COVID-19 patients and healthy subjects with a mass spectrometer. As a result, the inventors have found the amounts of 6-threonylcarbamoyl adenosine (t⁶A) and 2-thiomethyl,6-threonylcarbamoyl adenosine (ms²t⁶A) to be significantly higher in COVID-19 patients, and completed the present invention. The inventors of the present invention have further analyzed the amounts of t⁶A and ms²t⁶A in the plasma and urine of various febrile patients and revealed that their modified nucleosides were specifically higher in COVID-19 patients compared to other febrile patients. The inventors of the present invention have also found that the amounts of t⁶A and ms²t⁶A in patients were associated with subsequent changes in patient pathology.

The present invention includes the following modes.

[1] A method for determining whether a subject that is a mammal is at risk of suffering or is suffering from COVID-19, the method including: a step of detecting an amount of a modified nucleoside which is 6-threonylcarbamoyl adenosine (t⁶A) and/or 2-thiomethyl,6-threonylcarbamoyl adenosine (ms²t⁶A) in a sample derived from the subject; and a step of providing the amount of the modified nucleoside detected for the determination.

[2] The method according to [1], further including a step of determining whether the subject is at risk of suffering or is suffering from COVID-19 by comparing the amount of the modified nucleoside detected with a predetermined reference value.

[3] The method according to [1], further including a step of evaluating severity (degree of COVID-19 morbidity, for example, mild, moderate (moderate I, moderate II), severe) of the subject based on the amount of the modified nucleoside detected.

[4] The method according to [2], further including a step of predicting a subsequent change in pathology of the subject based on the amount of the modified nucleoside detected.

[5] A method for evaluating a therapeutic effect in a mammal suffering from COVID-19, the method including: a step of detecting an amount of a modified nucleoside which is 6-threonylcarbamoyl adenosine (t⁶A) and/or 2-thiomethyl,6-threonylcarbamoyl adenosine (ms²t⁶A) in a sample derived from a subject; and a step of providing the amount of the modified nucleoside detected for the evaluation.

[6] The method according to any one of [1] to [5], wherein the sample derived from a subject is plasma, serum, or urine.

[7] The method according to any one of [1] to [6], wherein the amount of the modified nucleoside is detected by mass spectrometry (preferably, tandem mass spectrometry (MS/MS)).

[8] The method according to [7], wherein the sample is a sample subjected to deproteinization and desalination.

[9] The method according to [7] or [8], wherein the sample is plasma or serum, and the step of detecting the amount of the modified nucleoside includes a step of correcting a measurement result with an amount of adenosine in the plasma or serum.

[10] The method according to [7] or [8], wherein the sample is urine, and the step of detecting the amount of the modified nucleoside includes a step of correcting a measurement result with an amount of at least one substance selected from the group consisting of creatinine, urea nitrogen, uric acid, adenosine, and 3-amino-3-carboxypropyl uridine (acp³U) in the urine.

[11] The method according to any one of [1] to [6], wherein the amount of the modified nucleoside is detected by an ELISA method.

[12] The method according to any one of [1] to [11], wherein the subject is a human.

[13] The method according to [12], wherein the subject is a febrile patient.

[14] A method for determining whether a subject that is a mammal is a therapeutic subject of COVID-19, the method including: a step of detecting an amount of a modified nucleoside which is 6-threonylcarbamoyl adenosine (t⁶A) and/or 2-thiomethyl,6-threonylcarbamoyl adenosine (ms²t⁶A) in a sample derived from the subject; and a step of providing the amount of the modified nucleoside detected for the determination.

[15] The method according to [14], wherein the sample derived from a subject is plasma, serum, or urine.

[16] The method according to [14] or [15], wherein the subject is a human.

[17] A severity marker or severity predictive marker of COVID-19, made of 6-threonylcarbamoyl adenosine (t⁶A) and/or 2-thiomethyl,6-threonylcarbamoyl adenosine (ms²t⁶A).

[18] Use of a modified nucleoside which is 6-threonylcarbamoyl adenosine (t⁶A) and/or 2-thiomethyl,6-threonylcarbamoyl adenosine (ms²t⁶A) detected in a sample derived from a subject that is a mammal, as a severity marker or a severity predictive marker of COVID-19.

[19] The use according to [18], wherein the subject is a human, and the sample is plasma, serum, or urine.

The present invention is also directed to a method for diagnosing whether a subject suffers from COVID-19 based on the result of the modified nucleoside detected by the method according to any one of [1] to [13]. Therefore, the present invention is also a diagnosis method including the following steps.
(a) A step of detecting the amount of a modified nucleoside (preferably, t⁶A and ms²t⁶A) which is 6-threonylcarbamoyl adenosine (t⁶A) and/or 2-thiomethyl,6-threonylcarbamoyl adenosine (ms²t⁶A) in a sample derived from a patient (preferably plasma, serum, or urine, more preferably deproteinized and desalinated plasma, serum, or urine); and
(b) a step of diagnosing whether the patient suffers from COVID-19 based on the amount of modified nucleoside detected in step (a).

The detection of the amount of the modified nucleoside in step (a) can be performed by mass spectrometry (preferably, tandem mass spectrometry (MS/MS)) or an ELISA method.

When the sample is plasma or serum, step (a) can provide more accurate detection by correcting the amount of the modified nucleoside detected with the amount of adenosine in plasma or serum, and when the sample is urine, step (a) can provide more accurate detection by correcting the amount of the modified nucleoside detected with the amount of at least one substance selected from the group consisting of creatinine, urea nitrogen, uric acid, adenosine, and 3-amino-3-carboxypropyl uridine (acp³U).

### Advantageous Effects of Invention

The method according to the present invention can determine whether a subject is at risk of suffering or is suffering from COVID-19.

### BRIEF DESCRIPTION OF DRAWINGS

FIG. 1 is a diagram showing the structure of 6-threonylcarbamoyl adenosine (t⁶A) in the left figure, and showing the structure of 2-thiomethyl,6-threonylcarbamoyl adenosine (ms²t⁶A) in the right figure.

FIG. 2 is a diagram showing the result of analysis of modified nucleosides in RNA of ACE 22 overexpressing HEK 293 cells infected with SARS-CoV2.

FIG. 3 is a diagram showing the t⁶A amount in serum (after serum adenosine correction) in the left figure, and showing the ms²t⁶A amount in serum (after serum adenosine correction) in the right figure.

FIG. 4 is a diagram showing the result of comparing urinary t⁶A amounts (after urinary acp³U correction) of COVID-19 patients with other febrile patients.

FIG. 5 is a diagram showing an ROC curve of the t⁶A amount corrected with acp³U.

FIG. 6 is a diagram showing the result of comparing urinary ms²t⁶A amounts (after urinary acp³U correction) of COVID-19 patients with other febrile patients.

FIG. 7 is a diagram showing an ROC curve of the ms²t⁶A amount corrected with acp³U.

FIG. 8 is a diagram showing the result of comparing detection of a modified nucleic acid (t⁶A and ms²t⁶A) in COVID-19 infected patients with healthy subjects. However, the correction using the internal standard was not performed. Data represent average value SEM. * represents P<0.05, ** represents P<0.01, and *** represents P<0.001.

FIG. 9 is a diagram showing the result of comparing subject samples in detection of a modified nucleic acid (t⁶A and ms²t⁶A) in COVID-19 infected patients with healthy subjects. Urine samples were acp³U corrected and serum samples were adenosine corrected. Data represent average value±SEM, and points indicate individual subjects. **** represents P<0.0001.

FIG. 10 is a diagram showing the result of comparing the detection of modified nucleic acids (t⁶A and ms²t⁶A) in asymptomatic or mild patients with severe COVID-19 infected patients.

FIG. 11 is a diagram showing the detection result of modified nucleic acids (ms²t⁶A) in COVID-19 infected patients at the time of hospitalization, and the result showing subsequent changes in pathology of the patients. Data represent average value±SEM. * represents P<0.0035.

### DESCRIPTION OF EMBODIMENTS

Hereinafter, the present invention will be described in detail by taking an exemplary embodiment as an example, but the present invention is not limited to the embodiment described below.

It should be noted that, unless otherwise noted in the text, all technical and scientific terms used herein have the same meaning as commonly understood by those skilled in the art to which the present invention belongs. In addition, any materials and methods equivalent or similar to those described herein can be used in the practice of the present invention as well. In addition, all publications and patents cited herein in connection with the inventions described herein constitute a part of this specification, for example, as indicating methods, materials, or the like that can be used in the present invention.

In the present specification, the description of “A to B” indicating a numerical range means a numerical range including A and B that are end points. The same applies to “A through B”.

COVID-19 is the name given to the disease that was responsible for the worldwide pandemic, a pneumonia of unknown cause that occurred in December 2019. SARS-CoV-2 is a virus name that causes the virus. In the present specification, the expression “COVID-19” is used when referring to a disease, and the expression “SARS-CoV-2” is used when referring to a virus. However, whichever name is used, if it is clear whether a disease or a virus is represented in the context, the meaning is understood.

In the present invention, a “subject” or a “patient” refers to any mammal, and includes, but is not limited to, humans; non-human primates, including non-human primates such as chimpanzees, other apes, and monkey species; livestock such as cattle, sheep, pigs, goats, and horses; domestic mammals such as dogs and cats; and small or laboratory animals, including rodents such as mice, rats, and guinea pigs, preferably humans. “Subject” or “patient” also includes adults, young children, and newborns.

“Sample” as used in the present invention is a sample derived from any subject that can contain a modified nucleoside. Although not particularly limited, a body fluid sample derived from a subject is preferably used as the sample. “Body fluid sample” is a sample of any liquid that can be isolated from the body of an individual, including, but not limited to, blood, plasma, serum, saliva, urine, tears, sweat, and the like. Preferably, the body fluid is plasma, serum, or urine. In one mode, the sample used in the present invention is human-derived.

In the present invention, the term “suffering from COVID-19” means that a subject that is a mammal is infected with SARS-CoV2, which is the causative virus of COVID-19 and can be determined to have COVID-19. In the present invention, the term “at risk of suffering from COVID-19” means that a subject, which is a mammal, is suspected to be infected with SARS-CoV2, which is the causative virus of COVID-19.

A subject determined to be suffering from or at risk of suffering from COVID-19 by the method according to the present invention can also be diagnosed as being COVID-19 in combination with other detection methods of COVID-19. Other detection methods include, but are not limited to, a PCR test, an antigen test, and preferably a PCR test.

In the present invention, that the subject is a therapeutic subject of COVID-19 means that the subject to be subjected to the detection method according to the present invention can be determined to be infected with SARS-CoV2, which is the causative virus of COVID-19, suffering from COVID-19, and in need of treatment.

The severity of COVID-19 is classified into mild, moderate (moderate I, moderate II), and severe with reference to, for example, the Ministry of Health, Labour and Welfare's published “Guide to Clinical Practice for Novel Coronavirus Infection”. In addition, it is possible to refer to the WHO clinical practice guideline, and guidelines published by the U.S. National Institutes of Health or each country.

The modified nucleoside, which is the detection subject, is 6-threonylcarbamoyl adenosine (t⁶A) and/or 2-thiomethyl,6-threonylcarbamoyl adenosine (ms²t⁶A). Detection of t⁶A and/or ms²t⁶A is not particularly limited as long as these modified nucleosides can be detected, but is preferably detected by mass spectrometry or an ELISA method.

“Mass spectrometry” or “MS” is an analysis technique for identifying a compound by its mass, and is a method for ionizing a sample to be analyzed by applying energy such as a high voltage to the sample, and filtering, detecting, and/or measuring ions based on a mass-to-charge ratio (m/z) of the ions. There are many types of mass spectrometers depending on the sample ionization method and detection method, and the mass spectrometer can be used without particular limitation as long as it can be used for detecting t⁶A and/or ms²t⁶A. MS, and various mass spectrometers such as TOF-MS and MALDI-TOF-MS, which are improved types of MS, are commercially available, and can be appropriately used in the present invention.

In the case of using mass spectrometry, detection can be performed with one mass spectrometer, but a tandem mass spectrometer (tandem MS/MS) in which two mass spectrometers are connected in tandem is preferably used.

Tandem MS/MS is a device in which two mass spectrometers (MS) are coupled in series and have a collision activation chamber between them. First, a sample is ionized by a first MS, only ions of a specific mass number are selected and guided to the collision activation chamber, and the sample is collided with an inert gas such as Xe (xenon). Thereafter, secondary ions (product ions) generated from the ions selected by the first MS are detected by a second MS.

Examples of the ionization method include electrospray ionization (ESI), atmospheric pressure chemical ionization (APCI), atmospheric pressure photoionization (APPI), electron ionization (EI), fast electron bombardment (FAB)/liquid secondary ionization (LSIMS), matrix-assisted laser desorption/ionization (MALDI), field ionization, field desorption, thermal spray/plasma spray ionization, and particle beam ionization. Those skilled in the art can appropriately select the ionization method based on the analyte to be measured, the type of the sample, the type of the detector, the selection of the positive ion mode or the negative ion mode, and the like. There is no particular limitation as long as t⁶A and/or ms²t⁶A can be detected, and the above method can be appropriately used.

Tandem MS/MS is typically performed using selective reaction monitoring (SRM). The selective reaction monitoring refers to operating a mass spectrometer so as to continuously detect only a signal amount of a specific product ion generated from a compound to be analyzed, instead of acquiring a product ion spectrum in multi-stage mass spectrometry of two or more stages. In SRM, tandem mass spectrometry may be spatial or temporal.

In the case of using mass spectrometry, a method is preferably used in which liquid chromatography (LC) or gas chromatography (GC), more preferably LC is coupled to the upstream of a mass spectrometer (MS or MS/MS), and a sample is separated by LC or GC, then introduced into a mass spectrometer, and analyzed. With LC or GC coupled to the upstream of the mass spectrometer, for example, even when a blood sample or a urine sample is used, satisfactory analysis can be performed.

Examples of the chromatography type that can be used for LC can include partition chromatography, normal phase liquid chromatography (NPLC), displacement chromatography, reverse phase liquid chromatography (RPLC), size exclusion chromatography, ion exchange chromatography, and affinity chromatography.

In general, the mass spectrometer includes a sample introduction unit, an ionization unit (ion source), a mass separation unit (analyzer), a detection unit (detector), a vacuum evacuation unit (vacuum pump), a device control unit/data processing unit (data system), and the like.

Examples of the analyzer used in the mass spectrometer include a triple quadrupole analyzer, an ion trap analyzer, and a time-of-flight analyzer, and the triple quadrupole analyzer or the quadrupole time-of-flight (QTOF) analyzer is preferable. From the viewpoint that commercially available instrument platforms advantageous for SRM assays often use a triple quadrupole analyzer, it is more preferable to perform tandem MS/MS using the triple quadrupole analyzer also in the detection method according to the present invention. The term “triple quadrupole” as used herein means not only a quadrupole but also a case where a multipole or a stacked electrode is used instead of a quadrupole as generally understood by those skilled in the art. In the detection of the present invention, the mass spectrometry may be performed in a negative ion mode or in a positive ion mode.

In the method according to the present invention, for the detection of t⁶A and/or ms²t⁶A, triple quadrupole LC/MS/MS with LC coupled to the upstream of the mass spectrometer is preferably used.

In the method according to the present invention, when t⁶A and/or ms²t⁶A are detected using mass spectrometry, it is preferable to deproteinize and/or desalinate the sample previously. These pretreatments make it possible to detect the target substance with high sensitivity and accuracy.

Methods of deproteinization generally include insolubilization (addition of an acid such as perchloric acid, trichloroacetic acid, or metaphosphoric acid, addition of an organic solvent miscible with water, such as acetone, acetonitrile, methanol, or ethanol, and heating/cooling) by denaturation of proteins, physical removal (ultrafiltration with a membrane filter (such as a centrifugal filtration device), dialysis with a dialysis tube, ultracentrifugation), and the like. In addition, deproteinization treatment can be performed by using a permeation limiting filler such as an internal-surface reversed-phase filler, a hybrid filler, or a hydrophilic polymer filler. Although t⁶A and/or ms²t⁶A are not limited as long as detection is not hindered, examples of a preferred method for deproteinization include deproteinization using an insolubilization method by protein denaturation with an organic solvent miscible with water, and for example, deproteinization using methanol. Methods of deproteinization treatment are known, and can be performed according to a conventional method. Although not particularly limited, for example, in the deproteinization treatment, ethanol or methanol is added to a sample (preferably, body fluid sample) in an amount of 0.2 to 20 times of the sample, preferably an amount of 1 to 5 times of the sample, the mixture is reacted for a time sufficient for protein denaturation (for example, 15 minutes), then centrifugation is performed under conditions sufficient for precipitating denatured proteins (for example, 12,000×g for 15 minutes), and a supernatant (organic solvent layer) is collected, whereby a deproteinized sample can be obtained. The deproteinized sample can be used for LC as it is or after being dried by a centrifugal evaporator or the like and dissolved in an appropriate solvent such as distilled water.

As a desalination method, a known desalination method used in analysis can be appropriately used. In addition, using the deproteinization method described above can serve as the desalination.

In one mode of the method according to the present invention, the amount of t⁶A and/or ms²t⁶A in the sample is detected based on the measurement result using a mass spectrometer. The amount of t⁶A and/or ms²t⁶A can be detected by performing correction with or without an internal standard, but can be detected more accurately by using the internal standard. Internal standards can include, but are not limited to, when the sample is a blood sample, for example, plasma or serum, adenosine in plasma or serum, and when the sample is urine, creatinine, urea nitrogen, uric acid, adenosine, and 3-amino-3-carboxypropyl uridine (acp³U) in urine. Correcting the measured amount of t⁶A and/or ms²t⁶A using these measured values of the internal standard substance can detect the target substance more accurately. The detection of the amount of t⁶A and/or ms²t⁶A may also be performed using a calibration curve prepared previously.

In the case of using a blood sample, for example, plasma or serum, and performing correction using serum adenosine as an internal standard, if the amount of t⁶A in the blood sample exceeds at least 3 times, preferably 5 times, more preferably 10 times the serum adenosine, it can be determined the subject from which the blood sample is derived suffers or is at risk of suffering from COVID-19. If the amount of ms²t⁶A in the blood sample exceeds at least 2 times, preferably 3 times, more preferably 5 times the serum adenosine, it can be determined the subject from which the blood sample is derived suffers from or is at risk of suffering from COVID-19.

In the case of performing correction using a urine sample and 3-amino-3-carboxypropyl uridine (acp³U) as an internal standard, if the amount of t⁶A in the urine sample exceeds at least 50 times, preferably 80 times, more preferably 100 times acp³U, it can be determined the subject from which the urine sample is derived suffers from or is at risk of suffering from COVID-19. If the amount of ms²t⁶A in the urine sample exceeds at least 5 times, preferably 8 times, more preferably 10 times acp³U, it can be determined the subject from which the blood sample is derived suffers from or is at risk of suffering from COVID-19.

The method according to the present invention need not perform correction (for example, correction using an internal standard) in detecting the amount of t⁶A and/or ms²t⁶A. In such a case, the amount of t⁶A and the average value of the amount of t⁶A in the subject not suffering from COVID-19 (for example, healthy subjects) are set previously, and if the amount in the sample derived from the subject is larger than these values, it can be determined the subject is suffering from or is at risk of suffering from COVID-19. The same applies to the case of detecting the amount of ms²t⁶A in the method according to the present invention.

In one mode of the method according to the present invention, the detection of the amount of t⁶A and/or ms²t⁶A in the sample can also be performed using an ELISA method. The ELISA method is a method in which a specific antibody is bound to a target antigen contained in a sample and is detected and quantified using an enzymatic reaction. In the method according to the present invention, detection can be performed using an antibody against t⁶A and/or an antibody against ms²t⁶A. As the antibody against the modified nucleoside, an antibody produced according to a conventional method can be used, and an antibody produced by contract or a commercially available antibody can also be used. The antibody can be either a polyclonal antibody or a monoclonal antibody, but is preferably a monoclonal antibody.

In the method according to the present invention, when the amount of t⁶A and/or ms²t⁶A is detected using the ELISA method, the detection can be performed according to a conventional method. For example, any of a direct method, an indirect method, a sandwich method, and a competitive method can be used, but the sandwich method is preferable.

In one mode of the invention, the determination method according to the present invention can be used to determine that a subject suffers from or is at risk of suffering from COVID-19. In one mode of the determination method according to the present invention, it can be determined whether a subject is at risk of suffering or suffering from COVID-19 by comparing the amount of t⁶A and/or ms²t⁶A measured in a sample of the subject with a predetermined reference value. The predetermined reference value is not limited to this, and examples of this can include a value of t⁶A and/or ms²t⁶A detected in healthy subjects (healthy subjects), or a value obtained by adding a numerical value that can effectively exclude a false positive to the above numerical value. If the amount of t⁶A and/or ms²t⁶A in the subject increases significantly relative to a cut-off value (reference value), it can be determined the subject suffers from or is at risk of suffering from COVID-19. Whether or not the value is significantly increased may be appropriately determined according to the sensitivity required for the detection method, and may be set to, for example, 2 times, 3 times, 5 times, 10 times, or the like the average value of healthy subjects.

The cut-off value can be appropriately set by those skilled in the art from the viewpoint of sensitivity, specificity, positive predictive value of morbidity (infection), negative predictive value of morbidity (infection), and the like. This example includes setting based on ROC curve analysis. Another known detection method for COVID-19 (for example, PCR method) is used to determine the positive or negative of the detection subject, and compare this result with the data in the case of measuring the same subject using the method according to the present invention. From this result, it is also possible to determine the cut-off value of the method according to the present invention.

In another mode of the present invention, the detection of t⁶A and/or ms²t⁶A can be used to determine the severity of a subject in addition to determining that the subject suffers from COVID-19. The severity of the subject can be determined to be, for example, but not limited to, mild, moderate, or severe. In one mode of the determination method according to the present invention, the severity of the subject can be determined by comparing the amount of t⁶A and/or ms²t⁶A measured in the sample of the subject with each reference value of the predetermined severity.

In another mode of the present invention, detection of t⁶A and/or ms²t⁶A can be used to evaluate a therapeutic effect in a subject suffering from COVID-19. The therapeutic effect in a subject can be evaluated, for example, by comparing the amount of t⁶A and/or ms²t⁶A measured in a sample of the subject before and after treatment. Based on the result, a treatment policy and a medication regimen for the subject can be determined or changed.

In another mode of the present invention, the detection result of t⁶A and/or ms²t⁶A can be used to determine a pathological change or prognosis of a subject suffering from COVID-19. More specifically, in the case of the subject suffering from COVID-19 with mild or moderate I, preferably mild, it can be predicted whether the subject will become severe or not based on the t⁶A and/or ms²t⁶A values measured in a sample of the subject. For example, it can be determined that the risk of the subject becoming more severe is higher as the measured numerical value is greater. Alternatively, if the amount of t⁶A and/or ms²t⁶A in the subject is significantly increased with respect to a predetermined cut-off value (reference value), it can be determined that the risk of the subject becoming more severe is high. Whether the amount is significantly increased may be appropriately determined according to the sensitivity required for the detection method. The cut-off value can be appropriately set by those skilled in the art from the viewpoint of sensitivity, specificity, predictive accuracy of severity, and the like.

This example includes setting based on ROC curve analysis. In addition, the pathological change or prognosis of the detection subject is monitored, and the result is compared with the data when the same subject is measured using the method according to the present invention. From this result, the cut-off value of the present method can be determined. Based on the result, a treatment policy and a medication regimen for the subject can be determined or changed. The regimen of a drug may be changed with reference to, for example, the Ministry of Health, Labour and Welfare's “Guide to Clinical Practice for Novel Coronavirus Infection”, the WHO clinical practice guideline, clinical practice guidelines published by U.S. National Institutes of Health or each country, and the like, and these guidelines and the like, including revised versions, are incorporated herein.

The present invention will be more specifically described with reference to the following examples, but the examples are merely illustrative of the present invention and do not limit the scope of the present invention at all.

### EXAMPLES

### (First Example) Comprehensive Analysis of Modified Nucleosides in SARS-CoV-2 Infected Cells

Modified nucleosides in mammalian cells infected with SARS-CoV-2 were analyzed as follows.

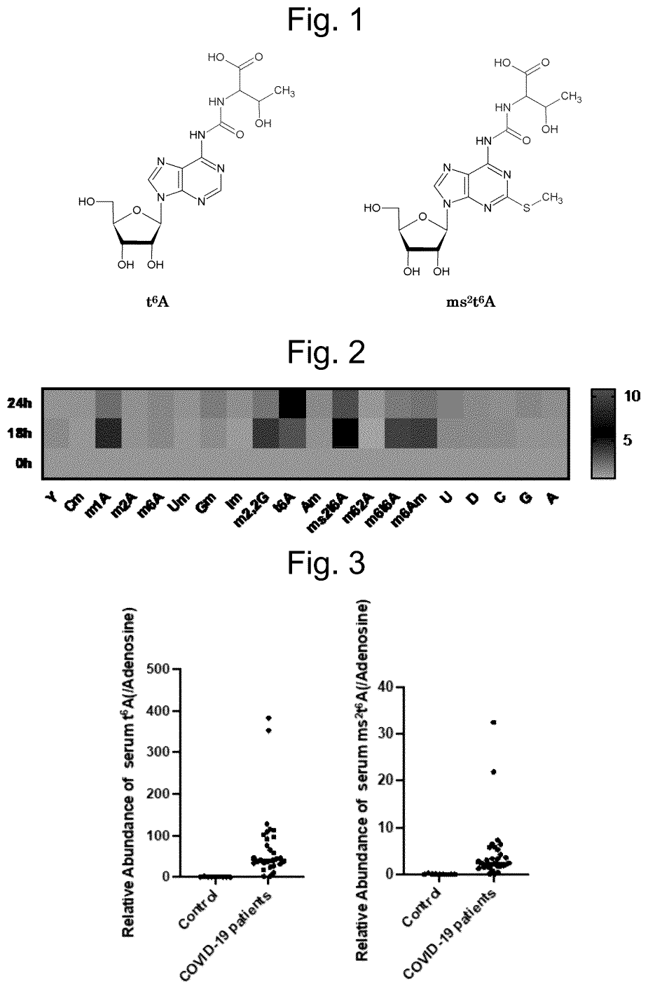

50 μL of a SARS-CoV2 solution (6×10⁶ VP/mL) was added to a cell culture solution (3×10⁵ cells) containing ACE2 overexpressing HEK293 cells to be infected, and RNA of the cells was extracted with Trizol at 18 hours and 24 hours after infection. The RNA concentration was adjusted to 1,000 ng/μL using a spectrophotometer (NanoDrop ND-1000). Thereafter, RNA degrading enzymes were used to degrade the nucleoside, and 2 μL of the sample was subjected to comprehensive analysis for modified nucleosides using an ultra-high speed triple quadrupole mass spectrometer (LCMS-8050 manufactured by Shimadzu Corporation). As a result, an increase in t⁶A and ms²t⁶A was observed in association with SARS-CoV2 infection. The respective structures are shown in FIG. 1. t⁶A increased 6 to 10 times with infection, and ms²t⁶A increased 3 to 5 times. The results are shown in FIG. 2.

### (Second Example) Detection of Modified Nucleic Acids in COVID-19 Infected Patients

Modified nucleic acids in COVID-19 infected patients were analyzed according to the following.

Serum and urine samples were collected from 30 patients diagnosed with COVID-19 at the time of hospitalization in a COVID-19 designated medical institution and stored in a freezer at −30° C. Thereafter, the sample was melted at normal temperature, and 100 μL of it was deproteinized and desalted using a column (Nanosep with 3K Omega). 2 μL of the sample was subjected to comprehensive analysis for modified nucleosides using an ultra-high speed triple quadrupole mass spectrometer (LCMS-8050 manufactured by Shimadzu Corporation). In addition, analysis was also performed by the same method for serum/urine of healthy subjects and urine of bacterial infected patients, influenza virus infected patients, and febrile patients after surgery. In addition, the measurement result in serum was corrected with the serum adenosine level, and the measurement result in urine was corrected with urinary 3-amino-3-carboxypropyl uridine (acp³U). The results using serum are shown in FIG. 3. The t⁶A amount and the ms²t⁶A amount in the COVID-19 patient serum were significantly larger than those of the healthy subjects.

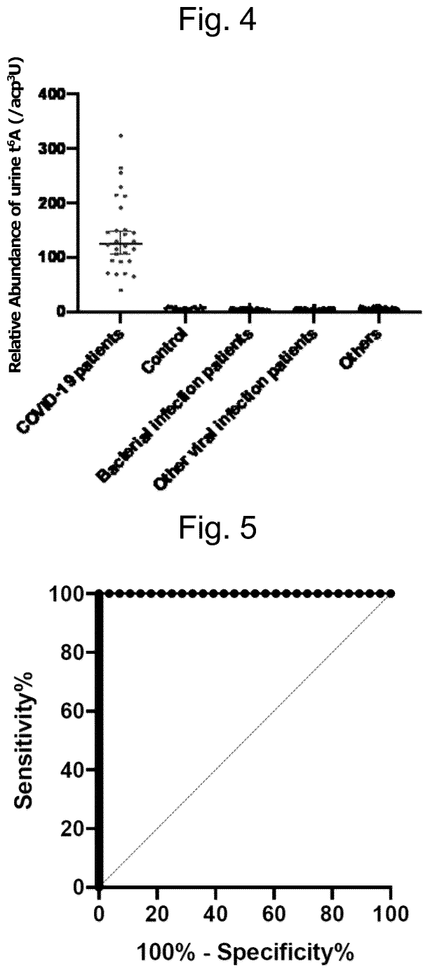

From the result of comparing and investigating the amounts of t⁶A in the urine of COVID-19 infected patients and each febrile patient, the amount of t⁶A in “Bacterial infection patients”, “Other viral infection patients” other than COVID-19 infected patients such as influenza patients, and “Others” who are febrile patients other than infectious disease patients such as patients having post-operative fever were equivalent to those in healthy subjects “Control”, but the amount of t⁶A in COVID-19 patients was significantly increased. The results are shown in FIG. 4.

An ROC curve of the amount of t⁶A in urine was drawn for COVID-19 patients, and sensitivity, specificity, and the like were investigated. When the cut-off value was set to 52.57, the sensitivity was 100%, the specificity was 96.43%, and the likelihood ratio was 28. The results are shown in FIG. 5.

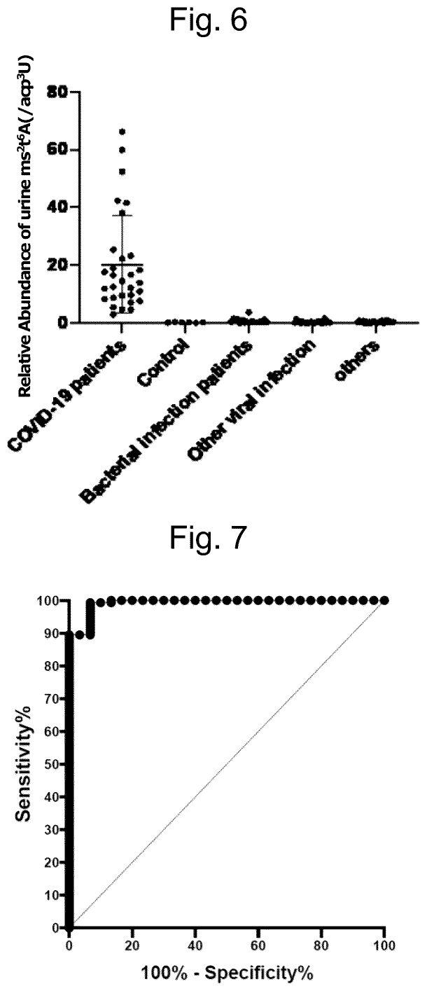

Next, from the result of comparing and investigating the amounts of ms²t⁶A in the urine of COVID-19 infected patients and each febrile patient, as with t⁶A, the amounts of ms²t⁶A in “Bacterial infection patients”, “Other viral infection patients” other than COVID-19 infected patients such as influenza patients, and “Others” who are febrile patients other than infectious disease patients such as patients having post-operative fever were equivalent to those in healthy subjects “Control”, but the amount of ms²t⁶A in COVID-19 patients was significantly increased. The results are shown in FIG. 6.

An ROC curve of the amount of ms²t⁶A in urine was drawn for COVID-19 patients, and sensitivity, specificity, and the like were investigated. When the cut-off value was set to 9.269, the sensitivity was 99.3%, the specificity was 93.33%, and the likelihood ratio was 14.9. The results are shown in FIG. 7.

### (Third Example) Detection of Modified Nucleic Acids in COVID-19 Infected Patients and Healthy Subjects (1)

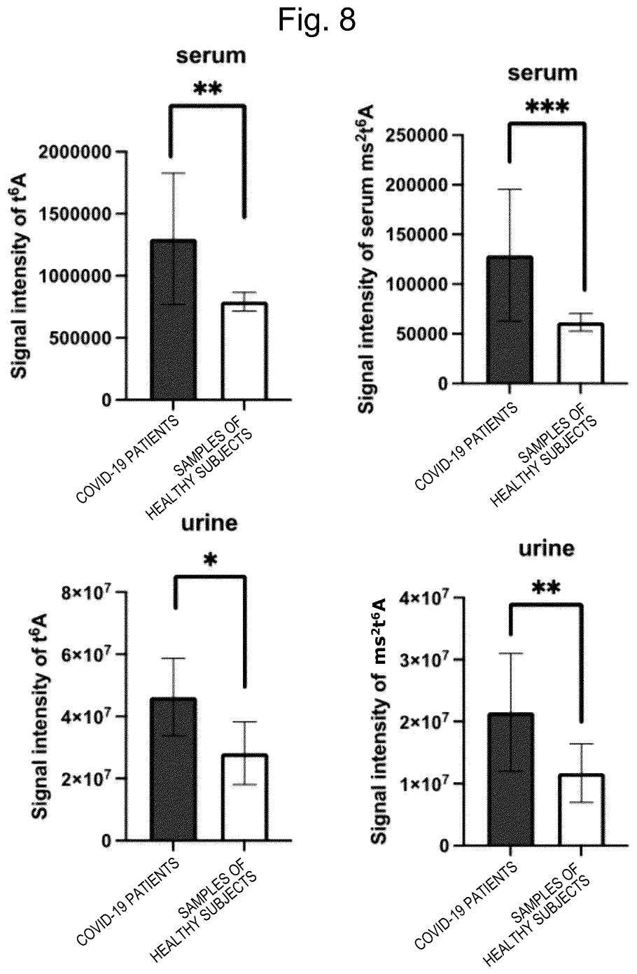

The t⁶A amount and the ms²t⁶A amount in serum and urine were measured using samples of COVID-19 infected patients (17 persons) and healthy subjects (14 persons) in the same manner as in the second example. The results are shown in FIG. 8. However, the correction using the internal standard was not performed. Even without correction, infection with COVID-19 in the subjects was able to be detected significantly.

### (Fourth Example) Detection of Modified Nucleic Acids in COVID-19 Infected Patients and Healthy Subjects (2)

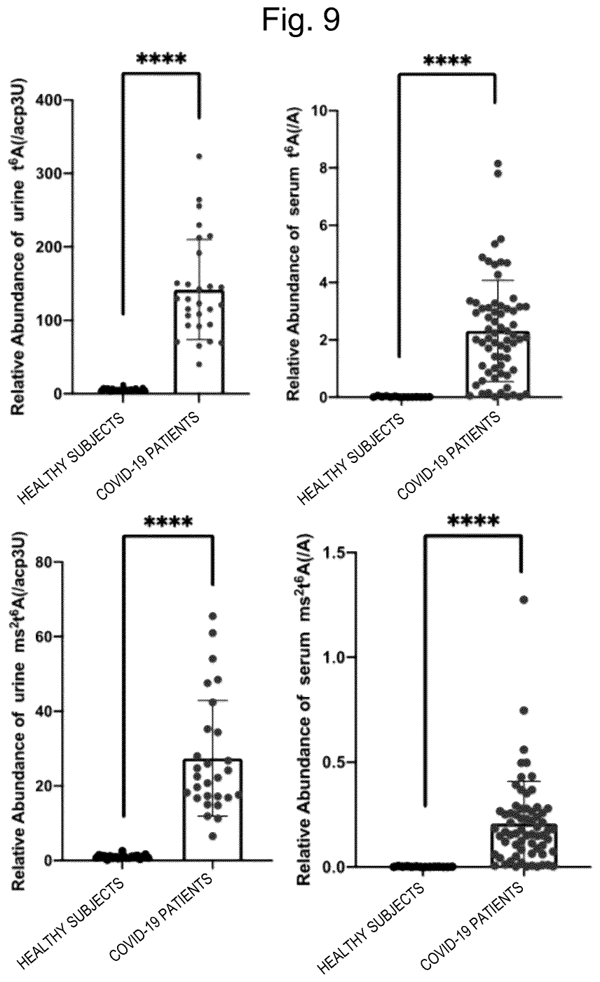

In the same manner as in the second example, the t⁶A amount and the ms²t⁶A amount in serum and urine were measured using samples of COVID-19 infected patients (28 persons) and healthy subjects (21 persons), and sensitivities in urine and serum were compared. The measurement result in serum was corrected with the serum adenosine level, and the measurement result in urine was corrected with urinary 3-amino-3-carboxypropyl uridine (acp³U). The results are shown in FIG. 9. In both the urine sample and the serum sample, infection with COVID-19 of the subject was able to be significantly detected in both the t⁶A amount and the ms²t⁶A amount, but the detection sensitivity was excellent when the urine sample was used.

### (Fifth Example) Detection of Modified Nucleic Acids in Patients with Severe COVID-19 Infection

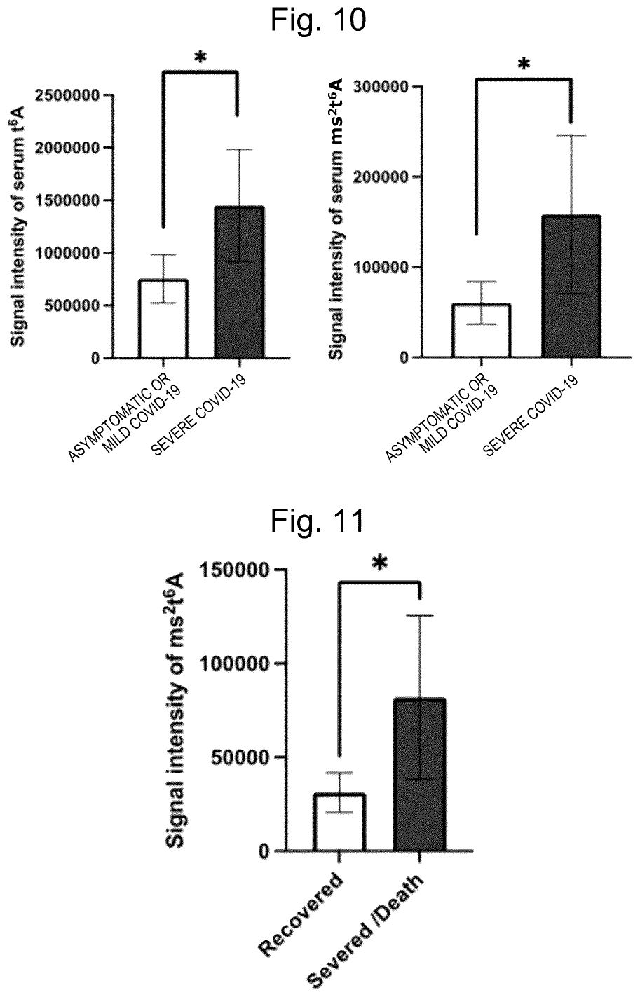

Using samples from asymptomatic or mild patients (58 patients) and severe patients (6 patients) with COVID-19 infection, the t⁶A amount and the ms²t⁶A amount in the serum were measured in the same manner as in the second example. However, the correction using the internal standard was not performed. The results are shown in FIG. 10. The amount of any modified nucleoside was significantly increased in the severe patients as compared with the asymptomatic patients and the mild patients. It has been found that the determination method according to the present invention can also be used to confirm a severe patient.

(Sixth Example) Prediction of pathological changes in COVID-19 infected patients Using samples of COVID-19 infected patients (21 patients) diagnosed as mild, the ms²t⁶A amount in the serum at the time of hospitalization was measured in the same manner as in the second example. However, the correction using the internal standard was not performed. Thereafter, the pathological change in each patient who received treatment was followed. 15 patients whose ms²t⁶A value was low at the time of hospitalization were discharged without exacerbation, whereas 6 patients whose ms²t⁶A value was high at the time of hospitalization became severe or died. Measurement results of the ms²t⁶A amount in the serum at the time of hospitalization of patients who were discharged from the hospital without exacerbation (Recovered) and patients who became severe or died (Severed/Death) are shown in FIG. 11. This result indicates that the prognosis of COVID-19 infected patients can be predicted by the marker and determination method according to the present invention.

### INDUSTRIAL APPLICABILITY

The present invention can determine whether a subject from which a sample is derived is at risk of suffering or suffering from COVID-19 by detecting a modified nucleoside in the sample, such as serum or urine, which can be easily obtained. Accordingly, the diagnosis of COVID-19 becomes simpler and easier. The method according to the present invention can also be used for determination of severity and prognosis of COVID-19.

1. A method for determining a treatment policy of a subject that is a mammal based on a result of determining whether the subject is at risk of suffering or is suffering from COVID-19, the method comprising:
a step of detecting an amount of a modified nucleoside which is 6-threonylcarbamoyl adenosine (t⁶A) and/or 2-thiomethyl,6-threonylcarbamoyl adenosine (ms²t⁶A) in a sample derived from the subject;
a step of determining whether the subject is at risk of suffering or is suffering from COVID-19 based on the amount of the modified nucleoside detected; and
a step of determining a treatment policy of the subject based on a result of the determination.

2. The method according to claim 1, further comprising a step of determining whether the subject is at risk of suffering or is suffering from COVID-19 by comparing the amount of the modified nucleoside detected with a predetermined reference value.

3. The method according to claim 1, further comprising a step of evaluating severity (degree of COVID-19 morbidity) of a subject based on the amount of the modified nucleoside detected.

4. The method according to claim 2, further comprising a step of predicting a subsequent change in pathology of the subject based on the amount of the modified nucleoside detected.

5. A method for determining a treatment policy of a subject that is a mammal suffering from COVID-19, the method comprising:
a step of detecting an amount of a modified nucleoside which is 6-threonylcarbamoyl adenosine (t⁶A) and/or 2-thiomethyl,6-threonylcarbamoyl adenosine (ms²t⁶A) in a sample derived from a subject;
a step of evaluating a therapeutic effect in the subject based on the amount of the modified nucleoside detected; and
a step of determining a treatment policy of the subject based on a result of the evaluation.

6. The method according to claim 1, wherein the sample is plasma, serum, or urine.

7. The method according to claim 1, wherein the amount of the modified nucleoside is detected by tandem mass spectrometry (MS/MS).

8. The method according to claim 7, wherein the sample is a sample subjected to deproteinization and desalination.

9. The method according to claim 7, wherein the sample is plasma or serum, and the step of detecting the amount of the modified nucleoside includes a step of correcting a measurement result with an amount of adenosine in the plasma or the serum.

10. The method according to claim 7, wherein the sample is urine, and the step of detecting the amount of the modified nucleoside includes a step of correcting a measurement result with an amount of at least one substance selected from the group consisting of creatinine, urea nitrogen, uric acid, adenosine, and 3-amino-3-carboxypropyl uridine in the urine.

11. The method according to claim 1, wherein the amount of the modified nucleoside is detected by an ELISA method.

12. The method according to claim 1, wherein the subject is a human.

13. The method according to claim 12, wherein the subject is a febrile patient.

14. A method for determining a treatment policy of a subject that is a mammal based on a result of determining whether the subject is a therapeutic subject of COVID-19, the method comprising:
a step of detecting an amount of a modified nucleoside which is 6-threonylcarbamoyl adenosine (t⁶A) and/or 2-thiomethyl,6-threonylcarbamoyl adenosine (ms²t⁶A) in a sample derived from the subject;
a step of determining whether the subject is a therapeutic subject of COVID-19 based on the amount of the modified nucleoside detected; and
a step of determining a treatment policy of the subject based on a result of the determination.

15. A method for determining a treatment policy of a subject that is a mammal suffering from COVID-19, the method comprising:
a step of detecting an amount of a severity marker or severity predictive marker of COVID-19, made of 6-threonylcarbamoyl adenosine (t⁶A) and/or 2-thiomethyl,6-threonylcarbamoyl adenosine (ms²t⁶A), in a sample derived from a subject;
a step of determining severity of the subject based on the amount of the severity marker or severity predictive marker detected; and
a step of determining a treatment policy of the subject based on a result of the determination.

16. The method according to claim 5, wherein the sample is plasma, serum, or urine.

17. The method according to claim 15, wherein the sample is plasma, serum, or urine.

18. The method according to claim 5, wherein the amount of the modified nucleoside is detected by tandem mass spectrometry (MS/MS).

19. The method according to claim 15, wherein the amount of the modified nucleoside is detected by tandem mass spectrometry (MS/MS).
